(19) United States
(12) Patent Application Publication
(10) Pub. No.: US 2006/0020250 A1
(43) Pub. Date: Jan. 26, 2006

(54) Absorbent structure

(75) Inventors: Stephen O. Chester, Greenville, SC (US); Samuel Charles Baer, Greenville, SC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: BBA Nonwovens Simpsonville, Inc.

(21) Appl. No.: 11/136,796

(22) Filed: May 25, 2005

Related U.S. Application Data
(60) Provisional application No. 60/590,840, filed on Jul. 23, 2004.
(60) Provisional application No. 60/606,559, filed on Sep. 2, 2004.

(51) Int. Cl. A61F 13/15

(52) U.S. Cl. 604/378

(57) ABSTRACT

An absorbent article is provided comprising a topsheet, a backsheet, and an absorbent core positioned between the topsheet and the backsheet, wherein the absorbent core comprises particles of superabsorbent polymer. An acquisition layer is positioned between the topsheet and the absorbent core for receiving and distributing fluid insults for absorption by the absorbent core. A temporary storage layer is positioned between the acquisition layer and the absorbent core, the temporary storage layer comprising a nonwoven fabric having a capacity to receive and temporarily hold the fluid insults in proximity to the absorbent core for a sufficient time for the fluid insults to be absorbed by the superabsorbent polymer in the absorbent core. In one advantageous embodiment, the acquisition layer has a calculated average pore size greater than 100 μm and the temporary storage layer has a calculated average pore size less than 100 μm.

### CROSS-REFERENCE TO RELATED APPLICATIONS

This application is related to and claims priority from U.S. Provisional Patent Application Nos. 60/590,840 filed Jul. 23, 2004 and 60/606,559 filed Sep. 2, 2004, the contents of which are incorporated herein by reference.

### FIELD OF THE INVENTION

This invention relates to absorbent articles, and more particularly absorbent structures which are useful in personal care products such as disposable diapers, child care training pants, incontinence garments and sanitary napkins.

### BACKGROUND OF THE INVENTION

Absorbent personal-care products of this type utilize an absorbent core for absorbing bodily fluids such as urine. Traditionally, the absorbent core has contained fiberized wood pulp as the primary absorbent. More recently, absorbent cores have used a blend of wood pulp and superabsorbent polymer (SAP) particles to reduce the thickness of the absorbent core. SAP particles are capable of absorbing many times their weight in liquid.

Manufacturers of absorbent personal care products have been looking for a “pulpless core” in order to simplify their manufacturing process by eliminating the need for complicated, costly fiberization equipment and to reduce raw material costs and price fluctuations typically associated with wood pulp. Several systems have been developed with this in mind, such as, for example, the laminate structure disclosed in U.S. Pat. No. 6,093,474 assigned to Korma S.p.A of Corso, Italy.

However, it has been discovered that when large amounts of SAP particles are used in the core, or when pulp is eliminated altogether from the core, the absorbent core is unable to absorb bodily fluids rapidly enough to prevent leakage. Surge layers such as those described in U.S. Pat. Nos. 5,879,343 or 5,994,615 have attempted to solve this problem but they still must include absorptive fibers such as pulp in order to function properly.

### BRIEF SUMMARY OF THE INVENTION

The absorbent system described herein solves the problems associated with the development of a pulpless absorbent core, and is useful in various absorbent articles using a pulp-free or low-pulp absorbent core.

In accordance with the present invention an absorbent article is provided comprising a topsheet, a backsheet, and an absorbent core positioned between the topsheet and the backsheet, wherein the absorbent core comprises particles of superabsorbent polymer. An acquisition layer is positioned between the topsheet and the absorbent core for receiving and distributing fluid insults for absorption by the absorbent core. A temporary storage layer (TSL) is positioned between the acquisition layer and the absorbent core, the temporary storage layer comprising a nonwoven fabric having a capacity to receive and temporarily hold the fluid insults in proximity to the absorbent core for a sufficient time for the fluid insults to be absorbed by the superabsorbent polymer in the absorbent core. The acquisition layer preferably comprises fibers or filaments defining a porous open structure with a porosity greater than that of the temporary storage layer. In one advantageous embodiment, the acquisition layer has a calculated average pore size greater than 100 μm and the temporary storage layer has a calculated average pore size less than 100 μm.

In a further embodiment of the present invention, the absorbent core is a pulp-free core which may suitably include a nonwoven fabric carrier layer with the particles of superabsorbent polymer adhered to the carrier layer. In one particular embodiment, the pulp-free core includes a rear carrier layer oriented toward the backsheet and a front carrier layer of nonwoven fabric oriented toward the topsheet, and wherein the superabsorbent polymer particles are trapped between said front and rear carrier layers. The structure of this general type is described in U.S. Pat. No. 6,093,474, which is incorporated herein by reference.

The absorbent core can be in the form of a single core layer, or it can be of a multi-layer construction. For example, the core may include a first absorbent core layer of a first size, and at least one additional absorbent core layer overlying the first absorbent core layer and being of a different size than the first absorbent core layer.

The temporary storage layer comprises at least one and desirably a plurality of layers of nonwoven fabric. The temporary storage layer may be attached to the front carrier layer of the absorbent core, or it may be incorporated into the absorbent core where it may serve as the front carrier layer of the absorbent core. In yet another embodiment, the temporary storage layer is separate from, but positioned in face-to-face relationship with the front carrier layer of the absorbent core. The nonwoven fabric in the temporary storage layer has a balance of porosity and wettability that allows it to distribute fluid within the temporary storage layer and to hold it in close proximity to the core and then to release the fluid to the core for absorption by the SAP particles.

The acquisition layer is designed for receiving fluid insults and for rapidly distributing the fluid over a larger area. The acquisition layer suitably includes a nonwoven fabric formed of fibers or filaments. Preferably, least one layer of nonwoven fabric in the temporary storage layer has fibers or filaments of a denier smaller than the fibers or filaments of the acquisition layer. It is also preferred that the temporary storage layer has a porosity (calculated average pore size) finer than that of the acquisition layer.

The drawings and detailed description which follows describes a pulpless absorbent core of the type described in the Korma U.S. Pat. No. 6,093,474. However, the system of the present invention will provide improvement to any type of pulpless core, especially one high in SAP particle content. The TSL must have a sufficient capacity to hold liquid and a small enough pore size to hold onto the liquid for a sufficient amount of time for the absorbent core to fully absorb the insult.

### BRIEF DESCRIPTION OF THE DRAWINGS

Having thus described the invention in general terms, reference will now be made to the accompanying drawings, which are not necessarily drawn to scale, and wherein:

FIG. 1 is a schematic top plan view of an absorbent article according to the present invention, with portions broken away to reveal the interior construction.

FIG. 2 is a cross-sectional view of the absorbent article of FIG. 1.

FIG. 3 is a cross-sectional view of an alternative embodiment of an absorbent article in accordance with the invention.

FIG. 4 is a schematic side view of the test stand for performing a runoff test.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The term “absorbent article” is used herein and a broad sense to include any article capable of receiving and absorbing fluids, and in particular body fluids. This term can be applied to baby diapers, adult incontinence diapers, sanitary napkins, panty liners, incontinence pads, and various other such articles.

The “calculated average pore size” of a nonwoven fabric may be determined according to the well-known formula, as published in the International Nonwoven Journal, Volume 6, No. 4, as follows:

$$Pore size , in microns = \begin{matrix}14.91 \sqrt{fiber denier \div batt density} \times \\ 11.89 \sqrt{fiber denier \div fiber density}\end{matrix}$$

where fiber denier is the number of weight in grams of 9000 meters of fiber, fiber density and batt density are in grams per cubic centimeter, and the batt density is measured under a compressive load of 14.7 grams per square centimeter (95 grams per square inch).

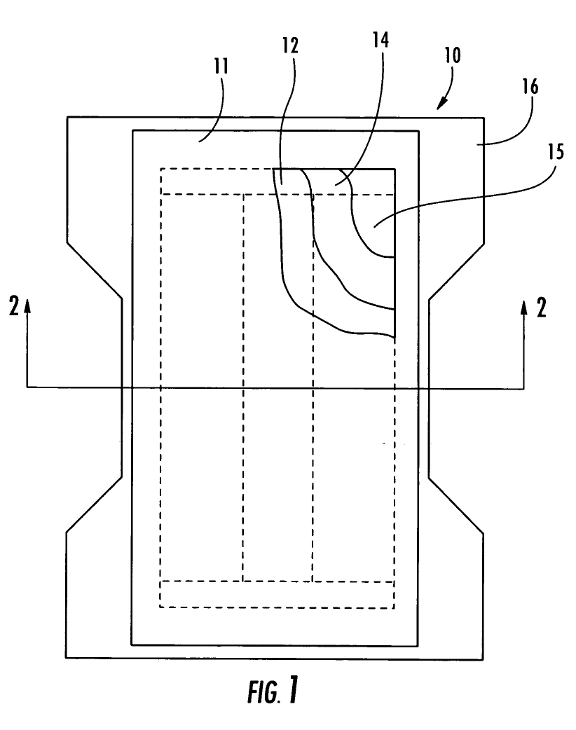

Referring now more particularly to the drawings and initially to FIG. 1, the reference character 10 indicates an absorbent article, which in the illustrated embodiment is in the form of a diaper. The absorbent article includes a topsheet 11 and acquisition/distribution layer 12 located beneath and directly in contact with the topsheet 11. Beneath the acquisition/distribution layer 12 there is provided a temporary storage layer or layers indicated at 14. The absorbent core 15 is positioned adjacent the temporary storage layer, and a fluid impermeable backsheet 16 underlies the core 15.

The topsheet 11 is designed to be positioned in contact with the wearer's body and may be formed of any of various kinds of materials conventionally used as topsheet material. Exemplary materials include nonwoven fabrics such as carded webs, air-laid webs, wet-laid webs, and spunbond webs. A suitable material is a soft polypropylene spunbond nonwoven fabric that contains a surfactant to render it wettable. The spunbond nonwoven fabric may have a basis weight of from about 10 to 30 grams per square meter (gsm). One example of a suitable topsheet fabric is a 14 gsm spunbond polypropylene nonwoven fabric which has been treated with a durable surfactant. The topsheet 11 can also be formed from an apertured thermoplastic film.

The acquisition layer 12 (sometimes referred to as an acquisition/distribution layer) is positioned adjacent to the topsheet 11 and is intended to initially acquire body fluids that pass through the topsheet and to transport and distribute these fluids over a larger area so that the full extent of the underlying absorbent core can be utilized for absorbing the fluids. To provide the rapid transport and distribution function, the acquisition layer preferably has a porous open structure. The acquisition layer 12 may suitably comprise one or more layers of a nonwoven fabric. Typically the nonwoven fabric of the acquisition layer 12 will have a calculated average pore size greater than 100 μm, and more typically from about 135 to 165 μm. The fibers used in the acquisition layer 12 can be hydrophilic, hydrophobic, or a combination of hydrophilic and hydrophobic fibers in order to provide the desired fluid transport properties. One example of a suitable acquisition layer is a 50 gsm carded resin bonded nonwoven fabric made from polyester (PET) fibers and having a calculated average pore size of 135 to 165 μm, and more specifically about 150 μm.

The backsheet layer 16 is a liquid impermeable material and can be made of any of the materials conventionally used for this layer, including nonwoven fabrics, films, and nonwoven fabric composites or laminates. It may, for example, comprise an apertured film or a vapor permeable, liquid impermeable film or nonwoven fabric composite.

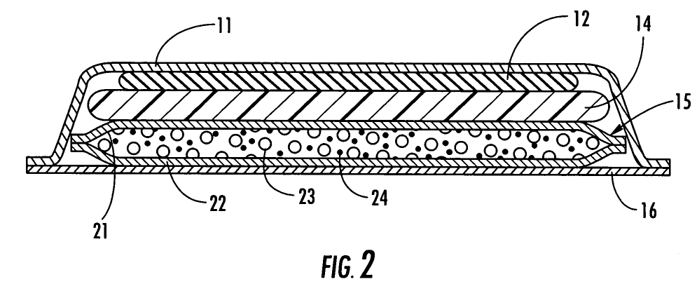

The absorbent core 15 can have various structures and shapes depending upon the specific design of the particular absorbent article. In the embodiment shown in the drawings, the absorbent core 15 is a single core structure of a generally rectangular shape. However, for certain types of absorbent products, the absorbent core can be a multilayer core with a first absorbent core layer of a first size, and at least one additional absorbent core layer overlying the first absorbent core layer and being of a different size (e.g. of smaller width and length dimensions) than the first absorbent core layer so as to provide additional absorption capacity in selected areas of the multilayer core. Although the present invention can be employed with conventional pulp-containing cores, the invention is especially advantageous with pulp-free absorbent cores. One such pulp-free core is described in U.S. Pat. No. 6,093,474 owned by Korma SpA, the contents of which are incorporated herein by reference. An absorbent core constructed as described in this patent is shown schematically in FIG. 2. It includes a front carrier layer 21, which may be formed from a porous nonwoven fabric such as a spunbond nonwoven, and which may suitably be treated with a surfactant to enhance wettability, and a rear carrier layer 22, which can be a porous nonwoven fabric or other porous or nonporous sheet material. Particles 23 of superabsorbent polymer (SAP) are deposited between the front and rear carrier layers 21, 22. SAP particles are commercially available from a number of sources, such as Dow, Degussa, BASF, and Atofina. A particulate thermoplastic adhesive may be mixed with the SAP particles so that the assembly can be thermally bonded together to form a unitary structure with the SAP particles 23 held between the front and rear carrier layers 21, 22. Exemplary thermoplastic adhesives include polyolefin polymers and copolymers, such as polyethylene, EVA copolymers, EAA copolymers, etc. Typically, the SAP particle-adhesive particle blend is provided in the absorbent core at a basis weight of about 250 to 450 grams per square meter, of which about 15-25% by weight is the thermoplastic adhesive particles and the balance is SAP particles. The carrier layers typically have a basis weight of about 10 to 70 grams per square meter.

The temporary storage layer 14 comprises at least one and preferably a plurality of layers of nonwoven fabric that have a capacity to receive the fluid insults from the acquisition layer 12 and to temporarily immobilize the fluid insults until the SAP particles can absorb the fluids. Unlike the acquisition layer 12, whose major function is to transport and distribute the fluids, the temporary storage layer 14 functions primarily to temporarily immobilize or hold the fluids in close proximity to the SAP particles and then to release the fluids into the core. This is achieved through a balance of the pore size of the nonwoven fabric structure and the wettability of the fibers. Preferably, the nonwoven fabric or fabrics used in the temporary storage layer 14 have a calculated average pore size of less than 100 μm, and more preferably less than about 70 μm. One particularly suitable fabric construction is a thermal bonded carded nonwoven fabric having a calculated average pore size of about 48 to 60 μm. The fiber size is typically within the range of 1.7 to 17 dtex (1.5 to 15 denier), with a size of about 2.2 to 2.2 dtex (2 to 3 denier) being preferred if the acquisition layer 12 is a nonwoven fabric and from 1.7 to 11 dtex (1.5 to 10 denier) if the acquisition layer is based upon an apertured film. A preferred density for a carded nonwoven fabric formed from 2.2 dtex (2 denier) polypropylene fibers is about 0.08 to 0.12 grams per cubic centimeter under a compressive load of 14.7 g/cm² (95 grams/square inch). The temporary storage layer 14 may comprise a layered structure with layers of differing fiber sizes.

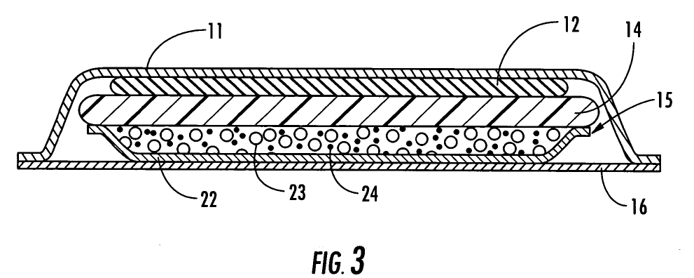

In the embodiment of the invention shown in FIG. 2, the temporary storage layer 14 is a separate individual component, and it is positioned overlying the absorbent core 15 and beneath the acquisition layer 12 during the fabrication of the absorbent article. In another embodiment, the temporary storage layer 14 can be affixed to the top carrier layer 21 of the absorbent core 15 with a suitable adhesive, such as a pressure sensitive adhesive or hot melt adhesive, so that these components can be handled as a unit during fabrication of the absorbent article. In yet another embodiment, as shown in FIG. 3, the temporary storage layer 14 can be incorporated into the structure of the absorbent core 15 as part of its uppermost carrier layer.

Various kinds of nonwoven fabrics can be used in producing the temporary storage layer and the fabrics can be made from fibers or filaments of various compositions. Examples of suitable nonwoven fabrics include carded webs, carded thermal bond webs, spunbond webs, air laid webs and wet laid webs. Particularly advantageous fluid handling properties can be achieved by using carded staple fiber webs that utilize fiber combinations of various sizes, polymer compositions and surface characteristics to improve the fluid penetration rate and short-term fluid retention of the temporary storage layer.

### EXAMPLES

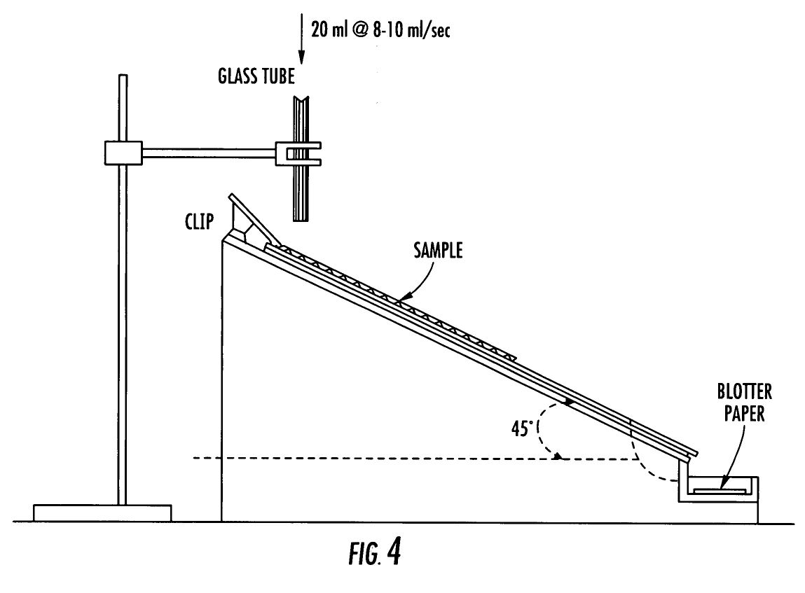

In order to ascertain the effectiveness of using a temporary storage layer, a modified run-off test was designed to simulate the mechanism of leakage of an absorbent article. 20 ml of 0.9% saline solution is discharged at a rate of about 8-10 ml/sec onto an absorbent system sample mounted onto a test stand tilted at an angle of 45 degrees from horizontal, as shown in FIG. 4. The liquid that is not absorbed by the sample is caught on pre-weighed blotter paper and weighed to determine the amount of “run-off” or leakage, from the sample. A total of three insults of 20 ml each are used in the test to further simulate real life usage of diapers. Sample size used was 10×20 cm (4 inch×8 inch) but can be varied according to the absorbent article being simulated.

Tests were conducted on a Korma-type pulp-free absorbent core system without a TSL and with various types of TSL's. The presence of the TSL clearly reduced run-out leakage by varying, but significant, amounts for most samples, as shown in the Table 1 below. The Korma-type pulpless absorbent core was constructed in accordance with the teachings of the above-noted U.S. Pat. No. 6,093,474 and has a front carrier layer formed from a 15 gsm spunbond polypropylene nonwoven fabric treated with a durable surfactant and a rear carrier layer formed from a 30 gsm spunbond polypropylene nonwoven fabric with no surfactant treatment. A blend of superabsorbent polymer (SAP) particles (Dow XZ91046.02) and EVA binder particles (18% by weight of the blend) are positioned between the front and rear carrier layers. The EVA binder particles serve to bond the core structure together. The blend of particles is present at a basis weight of 351 gsm.

TABLE 1

| TSL | Insult 1 | Insult 2 | Insult 3 | Total |
|---|---|---|---|---|
| None | 13.84 | 7.64 | 1.54 | 23.02 |
| 1 - 20 gsm carded | 10.96 | 3.70 | 0.00 | 14.66 |
| 2 - 20 gsm carded | 6.82 | 0.00 | 0.00 | 6.82 |
| 3 - 20 gsm carded | 5.47 | 0.00 | 0.00 | 5.47 |
| 1 - 30 gsm carded | 10.74 | 3.57 | 0.54 | 14.85 |
| 2 - 30 gsm carded | 6.07 | 0.86 | 0.00 | 6.93 |
| 1 - 15 gsm spunbond | 13.00 | 6.74 | 2.87 | 22.61 |
| 2 - 15 gsm spunbond | 11.67 | 4.76 | 2.39 | 18.82 |
| 3 - 15 gsm spunbond | 7.26 | 2.43 | 0.18 | 9.87 |
| 4 - 15 gsm spunbond | 6.88 | 1.44 | 0.00 | 8.32 |

The 15 gsm spunbond sample did not perform as well due to its overall low capacity or void volume. Note that as capacity or void volume was increased, the results improved. It is also believed that a relatively low pore size (as compared to the pore size of the acquisition layer) is preferable in the TSL since finer pores will tend to have a better ability to “hold onto” liquid. Durable hydrophilicity of the TSL fabric is also believed to help in handling the multiple insults.

One fabric that has been considered as a TSL top layer incorporated into a Korma core is a high-performance chemical bonded PET nonwoven layer (TSL Fabric #CB-1). This 60 gsm material is utilized to improve the fluid penetration rate to the Korma absorbent core laminate but also appears to yield good run-off performance. Another TSL top layer example for this project was a 60 gsm carded thermal bonded fabric (TSL Fabric #TB-1) with a pore structure and a fiber surface chemistry designed to give the best distribution and transmission of fluid to the SAP particles in the Korma absorbent core. The run off data for these specialty fabrics as the top Korma layer versus a Korma core with a 15 gsm spunbond fabric as the top layer is shown in Table 2. The impact of the top carrier fabric on run-off performance is quite significant. For example TB-1, the top carrier fabric for Sample KL-2, had a calculated average pore size of 54 μm.

TABLE 2

| | | | | 20 ml Run-off in ml (15 gsm | | | |
|---|---|---|---|---|---|---|---|
| | | | | SBPP TS/60 gsm CBPETAL)@1 | | | |
| | Top Carrier | Bottom | | minute interval between insults | | | |
| Sample | Layer | Carrier Layer | Absorbent Layer | 1^{st} | 2^{nd} | 3^{rd} | Total |
| KL-1 | SBPP | SBPP | SAP/Thermoplastic | 14.3 | 7.5 | 1.8 | 23.6 |
| | 15 gsm | 30 gsm | Powder Blend #1 | | | | |
| KL-2 | TB-1 | Carded PP | SAP/Thermoplastic | 2.8 | 0.1 | 0.1 | 3.0 |
| | (60 gsm) | 30 gsm | Powder Blend #1 | | | | |
| KL-3 | CB-1 | Carded PP | SAP/Thermoplastic | 3.9 | 1.4 | 1.0 | 6.3 |
| | (60 gsm) | 30 gsm | Powder Blend #1 | | | | |

Another set of Korma carrier fabrics utilize layered structures of large denier and small denier fibers. The large denier fibers provide void space for fluid penetration either within or on top of the Korma core while the small denier fibers provide powder containment and fluid distribution.

Another approach is to utilize fabrics of appropriate structure and surface characteristics to function as a temporary storage layer or TSL that is separate from the acquisition layer (AL). The AL has a high porosity which allows fluid to rapidly penetrate the topsheet; however, this same high porosity allows the fluid to also rapidly escape back through the topsheet before the SAP particles in the core can absorb the fluid. The TSL complements the AL function in that it holds fluid in proximity to the core so that the SAP particles have time to absorb it.

A series of spunbond and carded fabrics was produced and their performance was evaluated on top of a Korma core by means of the 45 degree run-off test The results in the Table 3 below show that a carded fabric TSL can significantly reduce the amount of run off from a Korma core diaper mock-up. The TSL data should also be related to the run-off data for sample KL-2 above where the TSL fabric, TB-1, forms the top layer of the Korma laminate.

TABLE 3
Run-Off in ml for Multiple 20 ml Saline Insults

| | 1^{st} | 2^{nd} | 3^{rd} | Cumulataive |
|---|---|---|---|---|
| Sample | Insult | Insult | Insult | Run-Off |
| No TSL Layer | 13.79 | 8.06 | 1.62 | 23.47 |
| 60 gsm | 7.11 | 1.16 | 0.00 | 8.27 |
| Carded TSL | | | | |
| 40 gsm | 8.96 | 2.20 | 0.13 | 11.29 |
| Carded TSL | | | | |
| 30 gsm | 9.50 | 1.83 | 0.20 | 11.53 |
| Carded TSL | | | | |
| 20 gsm | 11.32 | 3.96 | 1.35 | 16.63 |
| Carded TSL | | | | |

Test Stand Set-UP

| | |
|---|---|
| Topsheet: | 14 gsm SB |
| Acquisition Layer: | 50 gsm Chemical Bonded PET |
| TSL Layer | Various |
| Korma Core: | SAP/Thermoplastic Blend #2 with 15 gsm SBPP |
| | upper layer and 20 gsm SBPP lower layer |
| Run-off Angle: | 45 degrees |

A measure of fluid spread across samples tested in the run-off test is shown in the table below. The spread is a measure of the width of the fluid stain perpendicular to the direction of fluid application. The test set-up is the same as that for run-off test described in Table 3. The data show that the TSL is temporarily holding onto the fluid as it tries to run out of the sample. The wider spread, in combination with the run off data from Table 3, shows that the TSL is increasing the efficient utilization of the superabsorbent core. An unexpected result in the data was the TSL-only sample having a broader spread than the combined TSL/ADL sample.

TABLE 4
Test: Run-Off Spread
45 degree angle
Specimen size: 10 × 20 cm
3 Insults @ 20 ml each
Spread measured 10 cm down from top
Measurements are in cm

| | INSULT WIDTH, cm | | | |
|---|---|---|---|---|
| | No TSL/ADL | Only TSL | Only ADL | TSL/ADL |
| Insult 1 | 1.0 | 6.4 | 4.5 | 7.5 |
| Insult 2 | 1.3 | 8.5 | 5.4 | 9.0 |
| Insult 3 | 1.9 | 9.8* | 6.0 | 9.3 |
| Insult 1 | 1.3 | 8.4 | 4.5 | 7.5 |
| Insult 2 | 1.6 | 9.8* | 5.0 | 8.4 |
| Insult 3 | 2.0 | 10.5* | 5.3 | 9.2 |
| Insult 1 | 0.9 | 7.8 | 4.0 | 7.6 |
| Insult 2 | 1.8 | 10.0* | 5.1 | 8.9 |
| Insult 3 | 2.0 | 10.5* | 5.2 | 9.1 |

*Indicates that the spread reached the end of the sample

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An absorbent article comprising: a topsheet, a backsheet, an absorbent core positioned between the topsheet and the backsheet, said absorbent core comprising particles of superabsorbent polymer, an acquisition layer positioned between the topsheet and the absorbent core for receiving fluids from the topsheet and transferring the fluids for absorption by the absorbent core, and a temporary storage layer positioned between the acquisition layer and the absorbent core, the temporary storage layer comprising a nonwoven fabric having a capacity to receive and temporarily hold the fluids in proximity to the absorbent core so that the fluids can be absorbed by the superabsorbent polymer in said absorbent core.

2. The absorbent article of claim 1, wherein the acquisition layer comprises fibers or filaments defining a porous open structure with a porosity greater than that of the temporary storage layer.

3. The absorbent article of claim 2, wherein the acquisition layer has a calculated average pore size greater than 100 μm and the temporary storage layer has a calculated average pore size less than 100 μm.

4. The absorbent article of claim 1, wherein the temporary storage layer comprises at least one layer of nonwoven fabric with a fiber size within the range of 1.7 to 17 dtex.

5. The absorbent article of claim 4, wherein the temporary storage layer comprises a layered nonwoven structure with layers of differing fiber sizes.

6. The absorbent article of claim 1, wherein the absorbent core is free from fluff pulp.

7. The absorbent article of claim 1, wherein the absorbent core includes a nonwoven fabric carrier layer, and said particles of superabsorbent polymer are adhered to said carrier layer.

8. The absorbent article of claim 1, wherein the absorbent core includes a rear carrier layer oriented toward said backsheet, and a front carrier layer of nonwoven fabric oriented toward said topsheet, and wherein the superabsorbent polymer particles are trapped between said front and rear carrier layers.

9. The absorbent article of claim 8, wherein the front carrier layer comprises a carded thermal bonded nonwoven fabric.

10. The absorbent article of claim 8, wherein the front carrier layer comprises a bonded nonwoven fabric formed from a blend of fibers of at least two different fiber sizes.

11. The absorbent article of claim 8, wherein the front carrier layer is a multilayer composite nonwoven fabric.

12. The absorbent article of claim 8, wherein the temporary storage layer is attached to the front carrier layer of the absorbent core.

13. The absorbent article of claim 8, wherein the temporary storage layer serves as the front carrier layer of the absorbent core.

14. The absorbent article of claim 8, wherein the temporary storage layer is separate from, but positioned in face-to-face relationship with the front carrier layer of the absorbent core.

15. The absorbent article of claim 1, wherein the absorbent core is a multilayer core including a first absorbent core layer of a first size, and at least one additional absorbent core layer overlying the first absorbent core layer and being of a different size than the first absorbent core layer.

16. The absorbent article of claim 15, wherein said first absorbent core layer and said at least one additional absorbent core layer each include a rear carrier layer oriented toward said backsheet, and a front carrier layer of nonwoven fabric oriented toward said topsheet, and wherein the superabsorbent polymer particles are trapped between said front and rear carrier layers.

17. The absorbent article of claim 1, wherein the acquisition layer is a nonwoven fabric having an open porous structure and a calculated average core size greater than 100 μm, and wherein the nonwoven fabric of the acquisition layer includes hydrophilic fibers or filaments.

18. The absorbent article of claim 1, wherein the acquisition layer is a nonwoven fabric formed of fibers or filaments, and wherein said at least one layer of nonwoven fabric in the temporary storage layer has fibers or filaments of a size smaller than the fibers or filaments of the acquisition layer.

19. The absorbent article of claim 18, wherein the nonwoven fabric of the temporary storage layer has an average pore size less than about 70 μm.

20. The absorbent article of claim 1, wherein the temporary storage layer comprises at least one layer of a carded thermal bonded nonwoven fabric.

21. A pulp-free absorbent core for an absorbent article comprising a front carrier layer, a rear carrier layer, a blend of superabsorbent particles and a particulate thermoplastic adhesive disposed between the front and rear carrier layers with the adhesive serving to bond the front and rear carrier layers together, and a temporary storage layer positioned adjacent the front carrier layer, the temporary storage layer comprising at least one nonwoven fabric layer having a calculated average pore size less than 100 μm and a capacity to receive and temporarily hold the fluids in proximity to the absorbent core so that the fluids can be absorbed by the superabsorbent polymer in said absorbent core.

22. The absorbent core of claim 21, wherein the temporary storage layer is attached to the front carrier layer.

23. The absorbent core of claim 21, wherein the temporary storage layer is separate from the front carrier layer.

24. The absorbent core of claim 21, wherein the temporary storage layer serves as the front carrier layer.
